(19) United States
(12) Plant Patent Application Publication
(10) Pub. No.: US 2014/0245503 P1
(43) Pub. Date: Aug. 28, 2014

(54) Raspberry plant - Vajolet Plus cultivar

(50) ;

(71) Applicant: SANT'ORSOLA SOCIETA' COOPERATIVE AGRICOLA (Italian Farmers Cooperative), (US)

(72) Inventor: Aldo Telch, Faver (IT)

(73) Assignee: SANT'ORSOLA SOCIETA' COOPERATIVA AGRICOLA (Italian Farmers Cooperative), Pergine Valsugana (IT)

(21) Appl. No.: 13/815,390

(22) Filed: Feb. 27, 2013

(51) Int. Cl. A01H 5/00

(52) U.S. Cl. PLT/204

(57) ABSTRACT

The raspberry cultivar Vajolet Plus was obtained by crossing of two unknown hybrid raspberry at the Val di Cembra, Faver, in Trentino Region of Italy. The new cultivar is a primocane variety producing two crops per year, wherein the fruits ripen on current season's cane in late to very late and on previous season's cane early to medium. The resulting fruits are broad conical and medium red in coloration.

### GENUS AND SPECIES

Rubus idaeus L.

### VARIETY DENOMINATION

“Vajolet Plus”

### BACKGROUND OF THE NEW VARIETY

A new and distinct cultivar of red raspberry (Rubus idaeus L.) hereinafter referred to by the cultivar denomination “Vajolet Plus” is disclosed. The new variety “Vajolet Plus” was obtained from the cross of unknown individual raspberry plants by free pollination.

The crossing for the new cultivar occurred on year 2003 in Val di Cembra (Faver, Trento—Italy) and it has been subject to selection on year 2005 and then tested since year 2007. The characteristics of the new cultivar have been found stable and have been transmitted without change through succeeding (more than 6) asexual propagations.

### SUMMARY OF THE INVENTION

The most outstanding and distinguishing characteristics of the “Vajolet Plus” cultivar are hereunder reported.
primocane raspberry plant; growing the variety as floricane is to be preferred;
Good fruit colour and flavour;
Large and firm red berries.

The new cultivar can be propagated by: the use of suckers produced in a spawn-bed, root cutting and use of tissue culture techniques. The root cutting is to be preferred.

### BRIEF DESCRIPTION OF THE PHOTOGRAPHS

The new red raspberry cultivar “Vajolet Plus” is illustrated by the accompanying photographs showing typical specimens of the new variety in colour as true as reasonably possible to make the same colour illustrations of this character by conventional photographic procedures.

FIG. 1 is a view showing fruiting rows with the ripe and immature fruits borne on the tips.

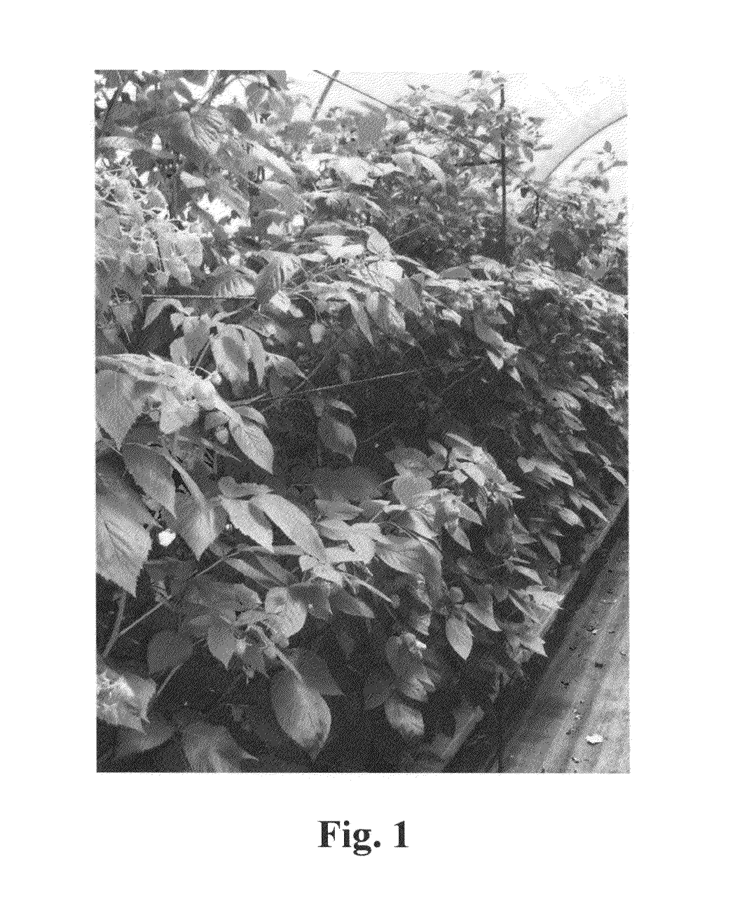

FIG. 2 is a view showing the fruit size, shape and colour at ripening.

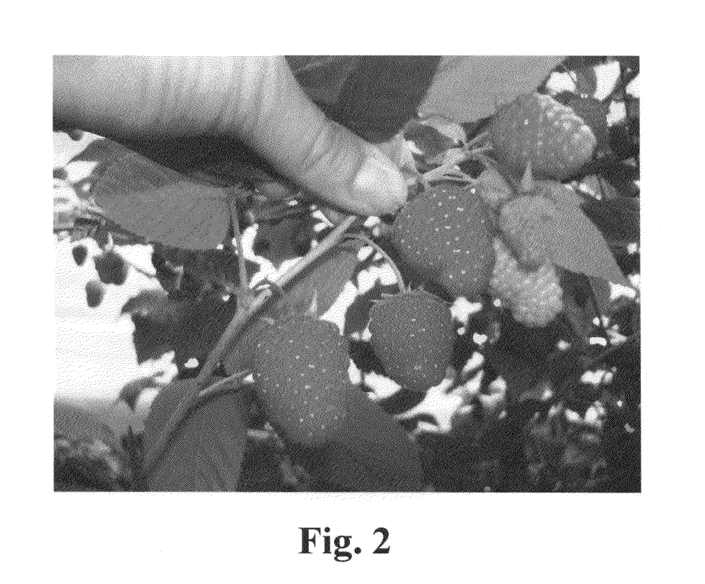

FIG. 3 is a view showing both the ripe and immature fruit size and shape.

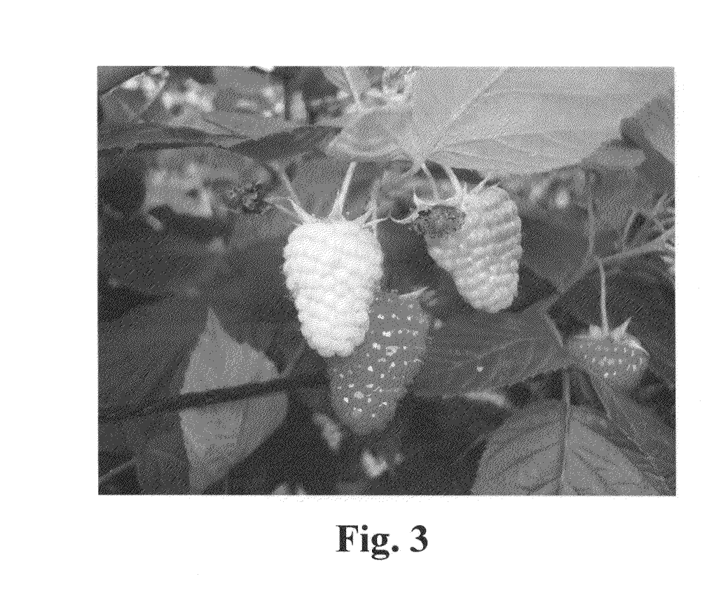

### DETAILED DESCRIPTION

The following detailed description set forth the distinctive characteristics of the new variety “Vajolet Plus”. The performance of new cultivar for retention of its distinctive characteristics has been evaluated through successive asexual propagations by root cutting in the Applicant's setting in Trentino (Italy) under controlled conditions of temperature (minimal temperature 15° C. and maximal temperature 28° C.) in greenhouse. The growing was the out of soil using peat as substrate.

The plant assessment was performed for 3 years.
Classification:
   Family.—Rosaceae.
   Botanical.—Rubus idaeus L.
   Common name.—Rasperry.
Parentage:
   Totally unknown.—
Plant:
   General habit.—upright canes; very strong vigor; very good roots system; adapted to lower-medium winter chill conditions such as south Italy; very high productivity in summer but not in autumn.
   Number of current season's shoots.—many to very many.
   Very young shoot.—anthocyanin coloration of apex during rapid growth present.
   Dormant cane colour.—brown.
   Spines.—present; the spines are dense, brownish purple.
   Leaves.—dark green on the superior surface and light green on the inferior surface; concave; odd-pinnate and subdivided in 3-5 ovate and serrate small leafs with strong rugosity.
   Bloom.—on the current season's cane is weak and the flowers are large and white and the blooming is late and begins in the late to very late season; on the previous season's cane is early and the flowers are large and white and the blooming is early to medium and begins in early to medium period.
Fruit:
   Predominant shape.—broad conical.
   Weight.—6-6.5 g (approximately).
   Size.—ratio length/width large.
   Colour of ripe fruit.—medium red.
   Firmness.—medium to firm.
   Main bearing time.—both on previous year's cane in summer and on current year's cane in autumn.
   Time of beginning the fruit ripening on previous season's cane.—early to medium.
   Time of beginning the fruit ripening on current season's cane.—late to very late.
   Storage capacity.—good shelf-life, good quality fruits adapted to fresh and frozen market.
Pest and disease resistance: Strong canes resistant to pests and diseases; the plants needs few spry treatments for the diseases and pests control. The new cultivar “Vajolet Plus” is similar to the commercial variety “Autumn Bliss” as shown in Table 1.

TABLE 1
Comparison between “Vajolet Plus” and “Autumn Bliss”

| Characteristic | “Vajolet Plus” | Autumn Bliss |
|---|---|---|
| Plant vigor | strong | medium |
| Roots | very strong | medium |
| Fruit shape | broad conical | conical |
| Productivity | high in summer | medium |
| Chill conditions | medium | lower |

What is claimed:

1. A new and distinct cultivar of raspberry plant as substantially herein shown and described.
